(19) United States
(12) Patent Application Publication
(10) Pub. No.: US 2025/0029256 A1
(43) Pub. Date: Jan. 23, 2025

(54) REDUCING NOISE AND ALIASING ARTEFACTS IN 4D MEDICAL IMAGES

(71) Applicant: Elekta Limited, Crawley (GB)

(72) Inventors: Samuele Papa, Crawley (GB); Jan-Jakob Sonke, Amsterdam (NL); Efstratios Gavves, Crawley (GB)

(21) Appl. No.: 18/762,196

(22) Filed: Jul. 2, 2024

(30) Foreign Application Priority Data
Jul. 20, 2023 (GB) 2311100.8

(51) Int. Cl. G06T 7/00; A61N 5/10

(52) U.S. Cl. CPC G06T 7/0014; A61N 5/1077; G06T 2207/10076; G06T 2207/20081; G06T 2207/30061

(57) ABSTRACT

Training an unsupervised Machine Learning model to reduce noise in a reconstructed 4D image can comprise obtaining a projection set comprising a plurality of 2D projections of a training patient volume, wherein the 2D projections represent a plurality of phases of a respiratory cycle of the training patient and comprise measurement noise, repeating, for a plurality of iterations, selecting two non-empty, mutually disjoint subsets that form a partition of the projection set, wherein each of the two selected subsets contains 2D projections that are respiratory uncorrelated for each of the two selected subsets, using a linear reconstruction algorithm to reconstruct a volumetric image of the training patient from the 2D projections contained in the subset, adding the reconstructed volumetric images to a training data set as an input volume and corresponding target output volume training pair, and using training pairs from the training data set to update values of trainable parameters of the ML model.

### CLAIM FOR PRIORITY

This application claims the benefit of priority of British Application No. 2311100.8, filed Jul. 20, 2023, which is hereby incorporated by reference in its entirety.

### TECHNICAL FIELD

The present disclosure relates to a method for training an unsupervised Machine Learning (ML) model to reduce noise and aliasing artefacts in a reconstructed four-dimensional (4D) medical image of a patient. The present disclosure also relates to a method for reducing noise and aliasing artefacts in a reconstructed 4D medical image of a patient. The present disclosure also relates to a training node, a processing node, and to a computer program product configured, when run on a computer, to carry out methods for training an unsupervised ML model and for reducing noise and aliasing artefacts in a reconstructed 4D medial image of a patient.

### BACKGROUND

Radiotherapy (RT) is one of the cornerstones of cancer treatment, using ionizing radiation to eradicate tumor cells. A total radiation dose for a patient can be divided into 3-30 daily fractions to optimize its effect. As the surrounding normal tissue is also sensitive to radiation, highly accurate delivery is a key part of effective RT. Image guided RT (IGRT) is a technique to capture the anatomy of the patient at the time of dose fraction delivery, using in-room imaging in order to align the treatment beam with the tumor location. Cone Beam Computed Tomography (CBCT) is the most widely used imaging modality for IGRT.

### SUMMARY

A major challenge, especially for CBCT imaging of the thorax and upper abdomen, is the respiratory motion that introduces blurring of the anatomy. Owing to the positional variation of the internal structures of the abdomen over the respiratory cycle of the patient, the localization accuracy and the sharpness of the image are reduced. A technique that can be used to alleviate motion artifacts is Respiratory Correlated CBCT (4DCBCT). From the 2D projections obtained during image acquisition, it is possible to extract a respiratory signal, which can indicate the position of the organs within the patient during the breathing cycle. Using this signal, subsets of the projections can be defined that relate to distinct phases of the respiratory cycle, enabling the creation of reconstructions that resolve the respiratory motion. However, as only 20 to 60 respiratory periods are imaged, the number of projections available for each phase of the respiratory cycle (and consequently each reconstruction) can be limited, and this limited number of projections for each reconstruction results in view-aliasing. Additionally, the projections are affected by stochastic measurement noise caused by the finite imaging dose used, and this further degrades the quality of the reconstruction, even when all projections are used.

Several methods based on iterative reconstruction algorithms and motion compensation techniques have been proposed to reduce view-aliasing in 4DCBCTs. While these methods reduce view-aliasing artifacts, they suffer from motion modeling uncertainty and prolonged reconstruction times. Deep learning has also been proposed to address view-aliasing with accelerated reconstruction times. Some techniques propose a supervised approach, which seeks to address view aliasing, but cannot reduce measurement noise, as this noise is still present in the images used as targets during training.

It is an aim of the present disclosure to provide methods, a training node, a processing node, and a computer program product which at least partially address one or more of the challenges mentioned above. It is a further aim of the present disclosure to provide methods, a training node, a processing node, and a computer program product which cooperate to enable the reduction of noise and aliasing artefacts in reconstructed 4D medical images of patients.

According to a first aspect of the present disclosure, there is provided a computer implemented method for training an unsupervised Machine Learning (ML) model to reduce noise and aliasing artefacts in a reconstructed four-dimensional (4D) medical image of a patient, wherein the ML model comprises a plurality of trainable parameters. The method comprises obtaining a projection set comprising a plurality of two-dimensional (2D) projections of a training patient volume, wherein the obtained 2D projections represent a plurality of phases of a respiratory cycle of the training patient, and wherein the obtained 2D projections comprise measurement noise. The method further comprises repeating, for a plurality of iterations, the steps of selecting two non-empty, mutually disjoint subsets that form a partition of the projection set, wherein each of the two selected subsets contains 2D projections that are respiratory uncorrelated, for each of the two selected subsets, using a linear reconstruction algorithm to reconstruct a volumetric image of the training patient from the 2D projections contained in the subset, and adding the reconstructed volumetric images of the patient to a training data set as an input volume and corresponding target output volume training pair. The method further comprises using training pairs from the training data set to update values of the trainable parameters of the ML model.

According to another aspect of the present disclosure, there is provided a computer implemented method for reducing noise and aliasing artefacts in a reconstructed 4D medical image of a patient. The method comprises obtaining a projection set comprising a plurality of 2D projections of the patient volume, wherein the obtained 2D projections represent a plurality of phases of a respiratory cycle of the patient, and wherein the obtained 2D projections comprise measurement noise. The method further comprises dividing the plurality of 2D projections in the projection set into a plurality of phase sets, wherein each phase set comprises 2D projections that are respiratory correlated, corresponding to a single phase of the respiratory cycle of the patient. The method further comprises, for each phase of the respiratory cycle of the patient, generating a reconstructed volumetric image of the patient from the 2D projections contained in the corresponding phase set, and inputting the generated reconstruction to an unsupervised ML model, wherein the ML model is operable to process the input generated reconstructed volumetric images according to trained parameters of the ML model, and to output volumetric images of the patient having reduced noise and aliasing artefacts. According to this aspect of the disclosure, the ML model has been trained using reconstructed volumetric images that are respiratory uncorrelated.

According to another aspect of the present disclosure, there is provided a computer program product comprising a computer readable medium, the computer readable medium having computer readable code embodied therein, the computer readable code being configured such that, on execution by a suitable computer or processor, the computer or processor is caused to perform a method according to any one or more aspects or examples of the present disclosure.

According to another aspect of the present disclosure, there is provided a training node for training an unsupervised ML model to reduce noise and aliasing artefacts in a reconstructed 4D medical image of a patient, wherein the ML model comprises a plurality of trainable parameters. The training node comprises processing circuitry configured to cause the training node to obtain a projection set comprising a plurality of 2D projections of a training patient volume, wherein the obtained 2D projections represent a plurality of phases of a respiratory cycle of the training patient, and wherein the obtained 2D projections comprise measurement noise. The processing circuitry is further configured to cause the training node to repeat, for a plurality of iterations, the steps of selecting two non-empty, mutually disjoint subsets that form a partition of the projection set, wherein each of the two selected subsets contains 2D projections that are respiratory uncorrelated, for each of the two selected subsets, using a linear reconstruction algorithm to reconstruct a volumetric image of the training patient from the 2D projections contained in the subset, and adding the reconstructed volumetric images of the patient to a training data set as an input volume and corresponding target output volume training pair. The processing circuitry is further configured to cause the training node to use training pairs from the training data set to update values of the trainable parameters of the ML model.

According to another aspect of the present disclosure, there is provided a processing node for reducing noise and aliasing artefacts in a reconstructed 4D medical image of a patient. The processing node comprises processing circuitry configured to cause the processing node to obtain a projection set comprising a plurality of 2D projections of the patient volume, wherein the obtained 2D projections represent a plurality of phases of a respiratory cycle of the patient, and wherein the obtained 2D projections comprise measurement noise. The processing circuitry is further configured to cause the processing node to divide the plurality of 2D projections in the projection set into a plurality of phase sets, wherein each phase set comprises 2D projections that are respiratory correlated, corresponding to a single phase of the respiratory cycle of the patient. The processing circuitry is further configured to cause the processing node to, for each phase of the respiratory cycle of the patient, generate a reconstructed volumetric image of the patient from the 2D projections contained in the corresponding phase set, and input the generated reconstruction to an unsupervised ML model, wherein the ML model is operable to process the input generated reconstructed volumetric images according to trained parameters of the ML model, and to output volumetric images of the patient having reduced noise and aliasing artefacts. According to this aspect of the disclosure, the ML model has been trained using reconstructed volumetric images that are respiratory uncorrelated.

According to another aspect of the present disclosure, there is provided radiotherapy treatment apparatus comprising at least one of a training node and/or a processing node according to any one of the aspects or examples of the present disclosure.

Aspects and examples of the present disclosure thus provide methods and nodes that are operable to reduce both noise and aliasing artefacts in 4D medical images.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document. For a better understanding of the present disclosure, and to show more clearly how it may be carried into effect, reference will now be made, by way of example, to the following drawings in which:

FIG. 1 illustrates an example of a flow chart illustrating process steps in a method for training an unsupervised ML model to reduce noise and aliasing artefacts in a reconstructed 4D medical image of a patient;

FIGS. 2A to 2C illustrate examples of flow charts illustrating process steps in another example of a method for training an unsupervised ML model to reduce noise and aliasing artefacts in a reconstructed 4D medical image of a patient;

FIG. 3 illustrates an example of a flow chart illustrating process steps in a method for reducing noise and aliasing artefacts in a reconstructed 4D medical image of a patient;

FIG. 4 illustrates an example of a flow chart illustrating process steps in another example of a method for reducing noise and aliasing artefacts in a reconstructed 4D medical image of a patient;

FIG. 5 illustrates an example of a block diagram illustrating functional modules in an example training node;

FIG. 6 illustrates an example a block diagram illustrating functional modules in an example processing node;

FIG. 7 illustrates an example of a qualitative comparison between methods using a coronal view of the patient in a test set;

FIG. 8 illustrates a Table showing a quantitative comparison between methods;

FIGS. 9A and 9B illustrates an example comparison of performance an implementation of the methods disclosed herein with different-sized datasets; and

FIG. 10 illustrates an example resolution of motion extent in an an implementation of the methods disclosed herein.

### DETAILED DESCRIPTION

Examples of the present disclosure propose methods enabling the reduction of both noise and aliasing artefacts in 4D medical images, such as 4DCBCT images. The present disclosure presents an example implementation of such methods, referred to as “Noise2Aliasing”, as well as demonstrating how the principles of the methods presented herein enable the provable training of models to reduce both aliasing artifacts and stochastic noise from 4DCBCTs in an unsupervised manner. The methods proposed in the present disclosure can be applied to pre-existing historical clinical datasets, and do not require any changes to current clinical practice.

As discussed in greater detail later in the present disclosure, the example implementation of the present methods, Noise2Aliasing, has been validated on publicly available data against a supervised approach, and has been applied to an internal clinical dataset of lung cancer patients. Different dataset sizes have been explored to understand their effects on the reconstructed images.

In order to provide additional context for the present disclosure, there now follows a brief introduction to concepts and notation relevant to understanding the methods presented herein, and to choices which may be made in their implementation.

Unsupervised Noise Reduction with Noise2Noise

The regression problem in the one-dimensional setting, which can be easily extended to multiple dimensions, consists in finding a function ƒ*: [symbol] → [symbol] which minimizes the Mean Squared Error (MSE) in expectation over the data. In this setting, input-target pairs (x, y) are available, with x, y∈ [symbol] , and can be used to find the desired function:

$${f}^{*} = {argmin}_{f} {𝔼}_{x , y} [ {‖ f ( x ) - y ‖}_{2}^{2} ] , \qquad Equation l$$

This can be minimized point-wise, resulting in the following:

$${f}^{*} ( x ) = {𝔼}_{y \vert x} [ y \vert x ] . \qquad Equation 2$$

(Noise2Noise) presents an approach, called “Noise2Noise”, in which input-target pairs are two samples of the same image. The samples only differ because of some independent mean-zero noise (x+δ₁, x+δ₂) with [symbol] _{δ}_{₂}[x+δ₂|x+δ₁]=x. Then ƒ* will recover the input image without any noise:

$${f}^{*} ( x + {δ}_{1} ) = {𝔼}_{{δ}_{2}} [ x + {δ}_{2} \vert x + {δ}_{1} ] = x . \qquad Equation 3$$

The removal of the noise from the input image depends upon the noise being both mean-zero, and element wise independent. Noise2Inverse seeks to extend this approach into the domain of volumetric imaging.

Denoising for Tomography with Noise2Inverse

During a CT scan, a volume x is imaged by acquiring 2D projections y=Ax using an x-ray source and a detector placed on the opposite side of the volume. The projections can then be used by an algorithm that computes a linear operator R to obtain an approximation of the original distribution of x-ray attenuation coefficients \hat{x}=Ry. The projections y are consequently the indirect measurements in 2 dimensions of the 3 dimensional volume x, and the measurements may be corrupted by elementwise independent measurement noise. Reconstruction of the volume x using the linear operator R causes elements of the measurement noise in the reconstructed volume to be coupled, meaning the noise in the reconstruction domain is no longer elementwise independent.

Noise2Inverse addresses the above issue by noting that the reconstruction algorithm can also operate on a subset of the projections. If [symbol] ={1, 2, . . . } is the set of all projections, and J⊂ [symbol] , then \hat{x}_{J}=R_{J}y_{J} is the reconstruction obtained using only projections y_{J}. It may be assumed that the projections have some mean-zero noise \tilde{y}ᵢ=yᵢ+∈ with [symbol] _{∈}(\tilde{y}ᵢ)=yᵢ. In Noise2Inverse, the results from Noise2Noise are extended to find a function ƒ* which removes projection noise when trained using noisy reconstructions \tilde{x}_{J}=R_{J}\tilde{y}_{J}=R_{J}y_{J}+R_{J}∈=\hat{x}_{J}+R_{J}∈ and the expected MSE as loss function. In particular, it is demonstrated that the loss function can be decomposed in the following way:

$$ℒ = 𝔼 {‖ f ( {\tilde{x}}_{{J}^{′}} ) - {\tilde{x}}_{J} ‖}_{2}^{2} = 𝔼 {‖ f ( {\tilde{x}}_{{J}^{′}} ) - {\hat{x}}_{J} ‖}_{2}^{2} + 𝔼 {‖ {\hat{x}}_{J} - {\tilde{x}}_{J} ‖}_{2}^{2} , \qquad Equation 4$$

Where J is a random variable that picks subsets of projections at random, and J′ is its complement.

Given Equation 2, it may be observed that function ƒ*, which minimizes [symbol] , is:

$${f}^{*} ( {\tilde{x}}_{{J}^{′}} ) = {𝔼}_{J} ( {\hat{x}}_{J} \vert {\tilde{x}}_{{J}^{′}} ) . \qquad Equation 5$$

When using reconstructions from a subset of noisy projections as input, and reconstructions from their complement as its output, a neural network will learn to predict the expected reconstruction without the noise.

### Property of Expectation Over Subsets of Projections Using the Iterative Algorithm FDK.

Let J be a random variable that selects subsets of projections J⊂ [symbol] at random such that each projection is selected at least once. Define R_{J}: [symbol] ^{D}^{_{d}}^{×|J|}→ [symbol] ^{D}^{ᵥ} to be the FDK reconstruction algorithm, which reconstructs a volume of dimensionality Dᵥ from projections J each with dimensionality D_{d} (geometrical details on the exact setup are not relevant). Then it is possible to see, thanks to the properties of the dual Radon, that the following holds:

$$\hat{x} = {R}_{𝒥} y = {𝔼}_{J \sim J} [ {R}_{J} {y}_{J} ] = {𝔼}_{J \sim J} [ {\hat{x}}_{J} ] . \qquad Equation 6$$

It will be appreciated that this holds in particular when the subsets of projections selected are a partition of [symbol] .

In effect, the Noise2Inverse algorithm addresses the issue of noise element coupling in the reconstruction domain by partitioning data in the projection domain, in which the measurement noise is element-wise independent. A Machine Learning (ML) model is then trained in the reconstruction domain to remove the noise. In each training step, the measured data (in the projection domain) is partitioned into an input component and a target component, and an ML model, such as a Convolutional Neural Network (CNN) is trained to predict the reconstruction generated from the output component using the reconstruction generated from the input component. After training, the CNN is applied to de-noise reconstructions generated from all available projections.

The authors of the present disclosure have determined that the approach of Noise2Inverse cannot naively be applied to 4DCBCT scans, as respiratory-correlated reconstructions do not correspond to the same clean underlying structure as can be expected from traditional CT scans. That is, the reconstructions of each phase of the respiratory cycle each represent a slightly different underlying physical structure, with the organs in a different position according to the phase of the respiratory cycle being represented. Additionally, 4DCBCT scans suffer from view aliasing, owing to the limited number of projections available for each reconstruction. Example methods according to the present disclosure present an approach that addresses these issues, enabling a reduction of noise, and of aliasing artefacts, in 4D medical images, such as 4DCBCT scans.

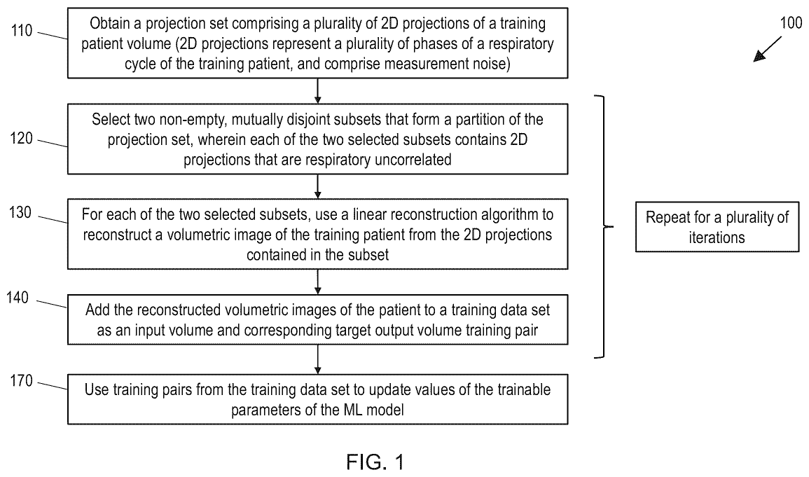

FIG. 1 is a flow chart illustrating process steps in a computer implemented method 100 for training an unsupervised Machine Learning (ML) model to reduce noise and aliasing artefacts in a reconstructed four-dimensional (4D) medical image of a patient, wherein the ML model comprises a plurality of trainable parameters. The method may be performed by a training node, which may comprise a physical or virtual node, and may be implemented in a computer system, treatment apparatus, such as a radiotherapy treatment apparatus, computing device, or server apparatus, and/or may be implemented in a virtualized environment, for example in a cloud, edge cloud, or fog deployment. Examples of a virtual node may include a piece of software or computer program, a code fragment operable to implement a computer program, a virtualised function, or any other logical entity. The training node may encompass multiple logical entities, as discussed in greater detail below.

Referring to FIG. 1, the method 100 comprises, in a first step 110, obtaining a projection set comprising a plurality of 2D projections of a training patient volume. As illustrated at step 110, the obtained 2D projections represent a plurality of phases of a respiratory cycle of the training patient, and the obtained 2D projections comprise measurement noise. The method 100 then comprises repeating, for a plurality of iterations, steps 120, 130, and 140. In step 120, the method 100 comprises selecting two non-empty, mutually disjoint subsets that from a partition of the projection set, wherein each of the two selected subsets contains 2D projections that are respiratory uncorrelated. In step 130, the method 100 comprises, for each of the two selected subsets, using a linear reconstruction algorithm to reconstruct a volumetric image of the training patient from the 2D projections contained in the subset. In step 140, the method 100 comprises adding the reconstructed volumetric images of the patient to a training data set as an input volume and corresponding target output volume training pair. Following the repetition of steps 120, 130, and 140 for the plurality of iterations, the method 100 further comprises, in step 170, using training pairs from the training data set to update values of the trainable parameters of the ML model.

According to the method 100, an unsupervised ML model is trained to reduce noise and aliasing artefacts in 4D patient images, using reconstructed training volumes that are reconstructed from subsets of projections of training patient volumes, those subsets containing respiratory uncorrelated projections. As discussed above, a 4D medical image of a patient comprises a collection of 3D images, each 3D image reconstructing the patient volume during a different phase of the respiratory cycle, so resolving the issue of respiratory motion during image acquisition. A 4D medical image is generated by collecting 2D projections of the patient volume over multiple respiratory cycles, before dividing the 2D projections into a plurality of bins, each bin corresponding to a different phase of the respiratory cycle, and containing projections that represent the internal structures of the patient volume during that phase of the cycle. Multiple 3D patient volumes are then reconstructed, each reconstructed volume using projections from a different bin, and representing patient anatomy in a different phase of the respiratory cycle. It will be appreciated that an important feature of the method 100 is that the input and target output training volumes used to train the ML model are generated from subsets of projections that are respiratory uncorrelated, that is projections that represent a plurality of different phases of the respiratory cycle. By generating training volumes using subsets containing a plurality of respiratory uncorrelated projections, it may be ensured that each of the training volumes represent the same underlying physical structure, which structure comprises the internal organs in their average position over the respiratory cycle.

It will be appreciated that the ML model of the method 100 is unsupervised, in that it is not trained via labelled target data that is free from noise and aliasing. On the contrary, the ML model is trained to map from a reconstruction generated using one subset of projections to a reconstruction generated using another non-overlapping subset. This enables the ML model when trained to reduce both mean-zero noise and aliasing artefacts.

The method may obtain the 2D projections either directly via an imaging apparatus such as CBCT, or the projections may be retrieved from a memory for training on historic patient data.

The method 100 is for training an unsupervised ML model. For the purposes of the present disclosure, the term “ML model” encompasses within its scope the following concepts:
machine Learning algorithms, comprising processes or instructions through which data may be used in a training process to generate a model artefact for performing a given task, or for representing a real-world process or system; and
the model artefact that is created by such a training process, and which comprises the computational architecture that performs the task.

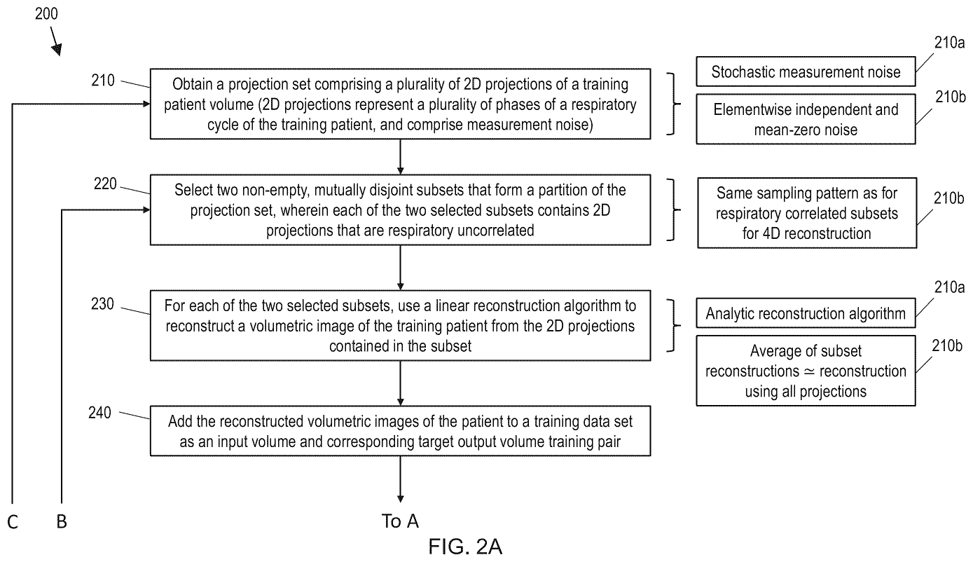

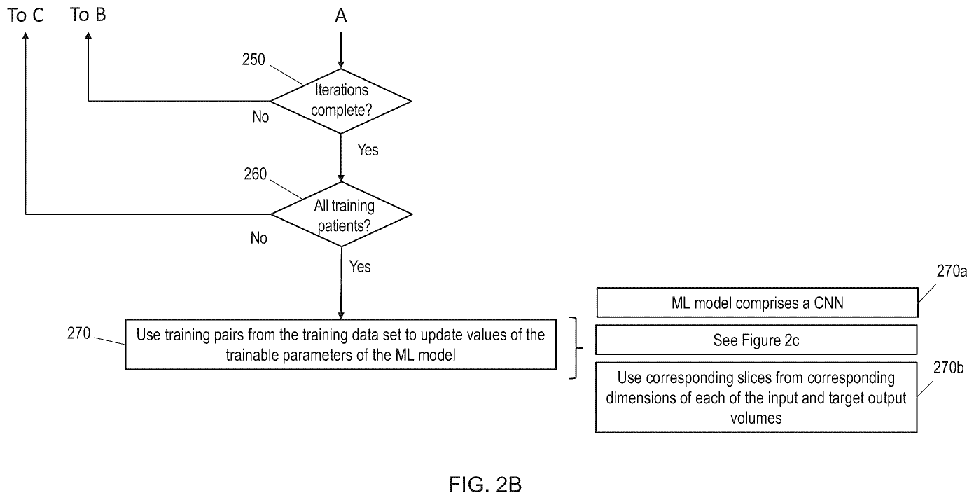

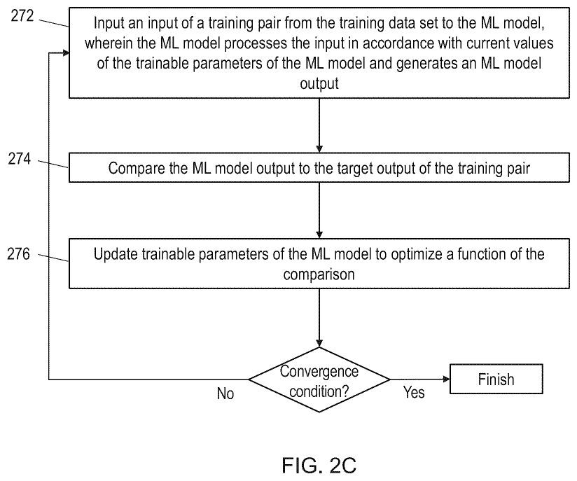

FIGS. 2A to 2C show flow charts illustrating a further example of a computer implemented method 200 for training an unsupervised ML model to reduce noise and aliasing artefacts in a reconstructed 4D medical image of a patient, wherein the ML model comprises a plurality of trainable parameters. As for the method 100 discussed above, the method 200 may be performed by a training node, which may comprise a physical or virtual node, and may be implemented in a computer system, treatment apparatus, such as a radiotherapy treatment apparatus, computing device, or server apparatus, and/or may be implemented in a virtualized environment, for example in a cloud, edge cloud, or fog deployment. Examples of a virtual node may include a piece of software or computer program, a code fragment operable to implement a computer program, a virtualised function, or any other logical entity. The training node may encompass multiple logical entities, as discussed in greater detail below. The method 200 illustrates an example of how the steps of the method 100 may be implemented and supplemented to provide the above discussed and additional functionality.

Referring initially to FIG. 2A, the training node first obtains a projection set comprising a plurality of 2D projections of a training patient volume in step 210. As illustrated at step 210, the obtained 2D projections represent a plurality of phases of a respiratory cycle of the training patient, and comprise measurement noise. As illustrated at 210a and 210b, the obtained 2D projections may comprise stochastic measurement noise, and may comprise measurement noise that is elementwise independent and mean-zero.

In step 220, the training node selects two non-empty, mutually disjoint subsets that form a partition of the projection set, wherein each of the two selected subsets contains 2D projections that are respiratory uncorrelated. As illustrated at 220a, this selecting step may comprise using a sampling pattern to select projections for inclusion in each of the two subsets, and the sampling pattern may be the same as a sampling pattern used to select projections for inclusion in respiratory correlated sets for reconstruction of a 4D medical image of a patient. According to examples of the present disclosure, the use of the same sampling pattern for selection of projections in respiratory uncorrelated subsets for training, and respiratory correlated subsets of 4D reconstruction, can ensure that the same aliasing effects are displayed in the training reconstructions as will be present in the respiratory correlated reconstructions of 4D images, meaning the model will be effective at removing these aliasing effects from the 4D images.

In step 230, for each of the two selected subsets, the training node uses a linear reconstruction algorithm to reconstruct a volumetric image of the training patient from the 2D projections contained in the subset. As illustrated at 230a, the reconstruction algorithm may comprise an analytic reconstruction algorithm. As illustrated at 230b, the reconstruction algorithm may have the property that an average of reconstructions generated by the algorithm using subsets of an available projection set is approximately equal to the reconstruction generated by the algorithm using all projections of the available projection set. For example, the reconstruction algorithm may comprise the FDK algorithm.

In step 240, the training node adds the reconstructed volumetric images of the patient to a training data set as an input volume and corresponding target output volume training pair.

Referring now to FIG. 2B, having added the reconstructed volumes to the training data set, the training node then checks, in step 250, whether a plurality of iterations of the steps 220, 230, and 240 have been completed for the current training patient. If the iterations have not been completed, the training node returns to step 220, and selects two new subsets forming a partition of the projection set, and repeats steps 230 and 240 for the newly selected subsets. If all of the iterations have been completed, then the training node then check, at step 260, whether all training patients have been considered. The method 200 may generate training data from a plurality of training patients. If all training patients have not yet been considered, then the training node returns to step 210, and obtains a set of 2D projections for a new training patient, before proceeding with steps 220 to 250 for the new training patient. Once all training patients have been considered, the training node proceeds to step 270, and uses training pairs from the training data set to update values of the trainable parameters of the ML model, which may be a Convolutional Neural Network (CNN), as illustrated at 270a.

FIG. 2C illustrates sub-steps that may be carried out by the training node in order to perform the step 270 of using training pairs from the training data set to update values of the trainable parameters of the ML model. Referring to FIG. 2C, the training node may repeat, until a convergence condition is satisfied, steps 272, 274 and 276. In step 272, the training node inputs an input of a training pair from the training data set to the ML model, wherein the ML model processes the input in accordance with current values of the trainable parameters of the ML model and generates an ML model output. In step 274, the training node compares the ML model output to the target output of the training pair. In step 276, the training node updates trainable parameters of the ML model to optimize a function of the comparison. According to examples of the present disclosure, the function of the comparison may comprise a function of the Mean Squared Error (MSE) between model output and target output.

The convergence criterion may for example comprise a threshold value of the function of the comparison (e.g., a threshold MSE), a threshold number of training iterations, a maximum or minimum training time, etc. A combined criterion may also be envisaged, in which some combination of threshold function value, training time and number or iterations is used, for example imposing a training stop after a certain maximum time, in the event that a threshold value for the function has not already been reached.

Referring again to FIG. 2B, in step 270, the training node may use training pairs from the training data set to update values of the trainable parameters of the ML model by using corresponding slices from corresponding dimensions of each of the input and target output volumes.

According to examples of the present disclosure, the methods 100, 200 may be complemented by s for reducing noise and aliasing artefacts in a reconstructed 4D medical image of a patient.

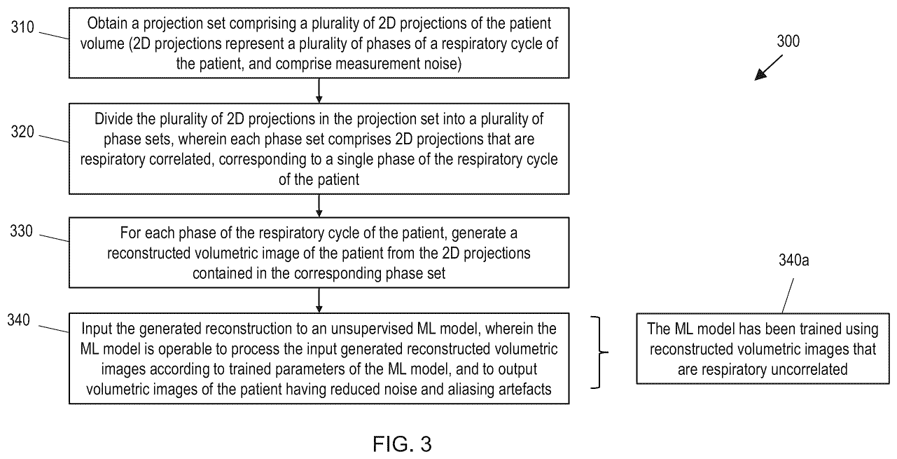

FIG. 3 is a flow chart illustrating process steps in a computer implemented method 300 for reducing noise and aliasing artefacts in a reconstructed 4D medical image of a patient. The method may be performed by a processing node, which may comprise a physical or virtual node, and may be implemented in a computer system, treatment apparatus, such as a radiotherapy treatment apparatus, computing device, or server apparatus, and/or may be implemented in a virtualized environment, for example in a cloud, edge cloud, or fog deployment. Examples of a virtual node may include a piece of software or computer program, a code fragment operable to implement a computer program, a virtualised function, or any other logical entity. The processing node may encompass multiple logical entities, as discussed in greater detail below.

Referring to FIG. 3, the method 300 comprises, in a first step 310, obtaining a projection set comprising a plurality of two-dimensional (2D) projections of the patient volume, wherein the obtained 2D projections represent a plurality of phases of a respiratory cycle of the patient, and wherein the obtained 2D projections comprise measurement noise. The method 300 further comprises, in step 320 dividing the plurality of 2D projections in the projection set into a plurality of phase sets, wherein each phase set comprises 2D projections that are respiratory correlated, corresponding to a single phase of the respiratory cycle of the patient. In step 330, the method 300 comprises, for each phase of the respiratory cycle of the patient, generating a reconstructed volumetric image of the patient from the 2D projections contained in the corresponding phase set. In step 340, the method 300 comprises inputting the generated reconstruction to an unsupervised M, model, wherein the ML model is operable to process the input generated reconstructed volumetric images according to trained parameters of the ML model, and to output volumetric images of the patient having reduced noise and aliasing artefacts. As illustrated at 340a, the ML model has been trained using reconstructed volumetric images that are respiratory uncorrelated.

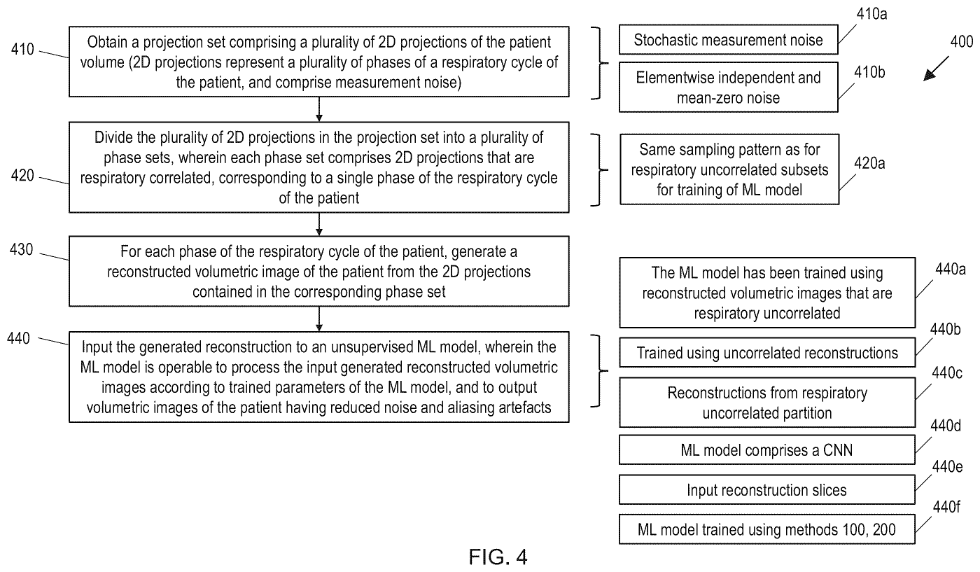

FIG. 4 is a flow chart illustrating process steps in another example of a method 400 for reducing noise and aliasing artefacts in a reconstructed 4D medical image of a patient. As for the method 300 discussed above, the method 400 may be performed by a processing node, which may comprise a physical or virtual node, and may be implemented in a computer system, treatment apparatus, such as a radiotherapy treatment apparatus, computing device, or server apparatus, and/or may be implemented in a virtualized environment, for example in a cloud, edge cloud, or fog deployment. Examples of a virtual node may include a piece of software or computer program, a code fragment operable to implement a computer program, a virtualised function, or any other logical entity. The processing node may encompass multiple logical entities, as discussed in greater detail below. The method 400 illustrates an example of how the steps of the method 300 may be implemented and supplemented to provide the above discussed and additional functionality.

Referring to FIG. 4, in step 410 the processing node obtains a projection set comprising a plurality of 2D projections of the patient volume, wherein the obtained 2D projections represent a plurality of phases of a respiratory cycle of the patient, and wherein the obtained 2D projections comprise measurement noise. As illustrated at 410a and 410b, the obtained 2D projections may comprise stochastic measurement noise, and may comprise measurement noise that is elementwise independent and mean-zero.

In step 420, the processing node divides the plurality of 2D projections in the projection set into a plurality of phase sets, wherein each phase set comprises 2D projections that are respiratory correlated, corresponding to a single phase of the respiratory cycle of the patient. As illustrated at 420a, the processing node may use a sampling pattern to select projections for inclusion in each of the phase sets, and the sampling pattern may be the same as a sampling pattern used to select projections for inclusion in respiratory uncorrelated sets for training of the ML model.

In step 430, for each phase of the respiratory cycle of the patient, the processing node generates a reconstructed volumetric image of the patient from the 2D projections contained in the corresponding phase set. The processing node may use an analytic reconstruction algorithm, such as FDK, for this step.

In step 440, the processing node inputs the generated reconstruction to an unsupervised ML model, wherein the ML model is operable to process the input generated reconstructed volumetric images according to trained parameters of the ML model, and to output volumetric images of the patient having reduced noise and aliasing artefacts. As illustrated at 440a, the ML model has been trained using reconstructed volumetric images that are respiratory uncorrelated.

As illustrated at 440b, the ML model may have been trained using training pairs of input and target output reconstructed volumetric images, wherein both the input and target output reconstructed volumetric images of a training pair are respiratory uncorrelated.

As illustrated at 440c, the input and target output reconstructed volumetric images of a training pair may be generated from two non-empty, mutually disjoint subsets that form a partition of a projection set for a training patient, and wherein each of the two subsets contains 2D projections that are respiratory uncorrelated.

As illustrated at 440d, the ML model may comprise a CNN.

As illustrated at 440e, inputting the generated reconstruction to an ML model may comprise inputting slices from the generated reconstruction to the ML model.

According to examples of the present disclosure, and as illustrated at 440f, the ML model may have been trained using an example method according to the present disclosure, such as the method 100 and/or 200.

As discussed above, an implementation of the methods disclosed herein, referred to as Noise2Aliasing, has been validated on clinical data, and experimental results are presented below. The efficacy of the methods presented herein may also be appreciated from a mathematical treatment of the problem. There now follows a mathematical examination of Noise2Aliasing, in which the FDK algorithm is used to generate reconstructed volumes for training in an implementation of the methods 100, 200.

Underlying Noise2Aliasing is the following proposition (which may be extended to other reconstruction algorithms as covered by the methods 100 to 400):

### Proposition:

Given the projection set [symbol] ={1, 2, . . . }, the FDK reconstruction algorithm R, and the noisy projections \tilde{y}=Ax+∈ with e mean-zero element-wise independent noise. Let J₁,J₂ be two random variables that pick different subsets at random belonging to a partition of [symbol] , and (\tilde{x}_{J}_{₁}=R_{J}_{₁}\tilde{y}_{J}_{₁},\tilde{x}_{J}_{₂}=R_{J}_{₂}\tilde{y}_{J}_{₂})∈ [symbol] be the input-target pairs in dataset [symbol] of reconstructions using disjoint subsets of noisy projections. Let [symbol] be the expected MSE over [symbol] with respect to a function ƒ: [symbol] ^{D}^{ᵥ}→ [symbol] ^{D}^{ᵥ} and the previously described input-target pairs. Then, the function ƒ* that minimizes [symbol] for any given J∈ [symbol] will reconstruct the volume using all the projections and remove the noise ∈:

$${f}^{*} ( {\tilde{x}}_{J} ) = \hat{x} \qquad Equation 7$$

### Proof:

The loss function [symbol] is defined in the following way:

$$ℒ = {𝔼}_{𝒟} {‖ f ( {\tilde{x}}_{{J}_{2}} ) - {\tilde{x}}_{{J}_{1}} ‖}_{2}^{2} . \qquad Equation 8$$

Additionally, J₁,J₂ are disjoint sets, the noise matches the characteristics required by Noise2Inverse (i.e., the noise is element-wise independent in the projection domain and is mean-zero), and the FDK reconstruction algorithm, which defines a linear operator R, is used. These assumptions allow use of Equation 5 to find that the function ƒ* that minimizes [symbol] is the following:

$${f}^{*} ( {\tilde{x}}_{J} ) = {𝔼}_{{J}_{1} , {J}_{2}} ( {\hat{x}}_{{J}_{1}} \vert {\tilde{x}}_{{J}_{2}} = {\tilde{x}}_{J} ) . \qquad Equation 9$$

Then, simplifying notation, and using the property of the FDK from Equation 6:

$$\begin{matrix}{f}^{*} ( z ) = {𝔼}_{{j}_{1} \sim {J}_{1}} [ {𝔼}_{{j}_{2} \sim {J}_{2}} ( {\hat{x}}_{{j}_{1}} \vert {\tilde{x}}_{{j}_{2}} = z ) ] \\ = {𝔼}_{{j}_{1} \sim {J}_{1}} ( {\hat{x}}_{{j}_{1}} ) = \hat{x} ,\end{matrix} \qquad Equation 10 and 11$$

Where \hat{x}ⱼ_{₁} is selected to be the clean reconstruction that is consistent with the observed noisy reconstruction z obtained from each disjoint subset j₂.

### Considerations on the Proposition.

Equation 11 holds true only when the same underlying clean reconstruction \hat{x} can be determined from the noisy reconstruction using any subset from a partition of the projections [symbol] . This means that, the training dataset should include reconstructions of the same underlying volume x using disjoint subsets of projections. In 4DCBCTs this is not the case, as separate respiratory phases are reconstructed, with the internal organs in different positions at each phase. As discussed above, examples of the present disclosure address this issue by selecting subsets of projections are respiratory uncorrelated, and consequently result in respiratory uncorrelated reconstructions. The reconstructions display organs in their average position (as opposed to their position during a specific phase of the respiratory cycle) meaning the respiratory uncorrelated reconstructions have the same underlying structure. In addition, if the projections are selected with the same sampling pattern as that used in respiratory-correlated reconstructions, then the view-aliasing artifacts displayed will have the same pattern as those present in the 4DCBCTs, improving the efficacy of aliasing artefact reduction in the 4DCBCT reconstructions. As discussed above the noise reduction effect of the trained ML model is achieved thanks to the selection of non-overlapping subsets of projections to generate the respiratory uncorrelated reconstructions.

Example methods according to the present disclosure achieve a reduction in noise and aliasing artefacts in 4DCBCT, so improving image quality without compromising on speed. The methods described above offer speed advantages associated with use of Deep Learning, and with the training, using currently available clinical data, of a model that is nonspecific to a particular patient, and consequently can be applied to improve image quality for a wide range of new patients. This combination of speed and quality can support both the planning and delivery of radiotherapy treatment, for example in the form of online Adaptive Radiotherapy (ART).

The speed and quality afforded by methods of the present disclosure can support the provision of online ART, in which in which CBCT is used to capture patient imaging at the start of each visit of the treatment fraction. This up-to-date imaging data, if available with sufficient quality, can enable clinicians to track changes in patient anatomy, including for example tumour shrinkage over the course of the radiotherapy treatment, allowing for online target localisation and plan adaptation without the constraints of diagnostic CT imaging. The improved image quality offered by methods according to the present disclosure may result in many additional medical treatment benefits (including improved accuracy of radiotherapy treatment, reduced exposure to unintended radiation, reduced treatment duration, etc.). The methods presented herein may be applicable to a variety of medical treatment and diagnostic settings or radiotherapy treatment equipment and devices.

In one particular use case for methods of the present disclosure, a dose from a previous treatment session can be deformed or modified in light of the current patient anatomy as represented by the reconstructed volumetric image of the patient. The output of the methods disclosed herein may thus be used in the creation or adaptation of a radiotherapy treatment plan.

As discussed above, the methods 100 and 200 may be performed by a training node, and the present disclosure provides a training node that is adapted to perform any or all of the steps of the above discussed methods. The training node may comprise a physical or virtual node, and may be implemented in a computer system, treatment apparatus, such as a radiotherapy treatment apparatus, computing device, or server apparatus, and/or may be implemented in a virtualized environment, for example in a cloud, edge cloud, or fog deployment. Examples of a virtual node may include a piece of software or computer program, a code fragment operable to implement a computer program, a virtualised function, or any other logical entity. The training node may encompass multiple logical entities, as discussed in greater detail below.

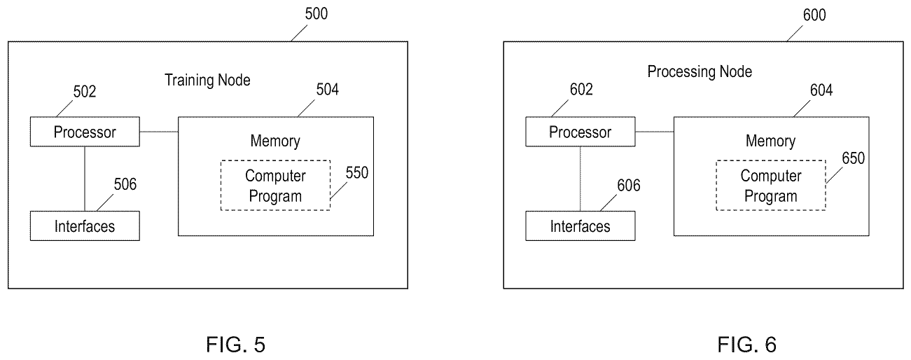

FIG. 5 is a block diagram illustrating an example training node 500 which may implement the method 100 and/or 200, as illustrated in FIGS. 1 to 2C, according to examples of the present disclosure, for example on receipt of suitable instructions from a computer program 550. Referring to FIG. 5 the training node 500 comprises a processor or processing circuitry 502, and may comprise a memory 504 and interfaces 506. The processing circuitry 502 is operable to perform some or all of the steps of the method 100 and/or 200 as discussed above with reference to FIGS. 1 to 2C. The memory 504 may contain instructions executable by the processing circuitry 502 such that the training node 500 is operable to perform some or all of the steps of the method 100 and/or 200, as illustrated in FIGS. 1 to 2C. The instructions may also include instructions for executing one or more telecommunications and/or data communications protocols. The instructions may be stored in the form of the computer program 550. In some examples, the processor or processing circuitry 502 may include one or more microprocessors or microcontrollers, as well as other digital hardware, which may include digital signal processors (DSPs), special-purpose digital logic, etc. The processor or processing circuitry 502 may be implemented by any type of integrated circuit, such as an Application Specific Integrated Circuit (ASIC). Field Programmable Gate Array (FPGA) etc. The memory 504 may include one or several types of memory suitable for the processor, such as read-only memory (ROM), random-access memory, cache memory, flash memory devices, optical storage devices, solid state disk, hard disk drive, etc. The interfaces 506 may be operable to communicate with other nodes or systems, including for example a processing node 600.

In some examples as discussed above, the training node may be incorporated into treatment apparatus, and examples of the present disclosure also provide a treatment apparatus comprising either or both of a training node as discussed above and/or a processing node as discussed below, and/or a planning node operable to implement a method for developing and/or adapting a radiotherapy treatment plan.

As discussed above, the methods 300 and 400 may be performed by a processing node, and the present disclosure provides a processing node 600 that is adapted to perform any or all of the steps of the above discussed methods. The processing node may comprise a physical or virtual node, and may be implemented in a computer system, treatment apparatus, such as a radiotherapy treatment apparatus, computing device, or server apparatus, and/or may be implemented in a virtualized environment, for example in a cloud, edge cloud, or fog deployment. Examples of a virtual node may include a piece of software or computer program, a code fragment operable to implement a computer program, a virtualised function, or any other logical entity. The processing node may encompass multiple logical entities, as discussed in greater detail below.

FIG. 6 is a block diagram illustrating an example processing node 600 which may implement the method 300 and/or 400, as illustrated in FIGS. 3 and 4, according to examples of the present disclosure, for example on receipt of suitable instructions from a computer program 650. Referring to FIG. 6 the processing node 600 comprises a processor or processing circuitry 602, and may comprise a memory 604 and interfaces 606. The processing circuitry 602 is operable to perform some or all of the steps of the method 300 and/or 400 as discussed above with reference to FIGS. 3 and 4. The memory 604 may contain instructions executable by the processing circuitry 602 such that the processing node 600 is operable to perform some or all of the steps of the method 300 and/or 400, as illustrated in FIGS. 3 and 4. The instructions may also include instructions for executing one or more telecommunications and/or data communications protocols. The instructions may be stored in the form of the computer program 650. In some examples, the processor or processing circuitry 602 may include one or more microprocessors or microcontrollers, as well as other digital hardware, which may include digital signal processors (DSPs), special-purpose digital logic, etc. The processor or processing circuitry 602 may be implemented by any type of integrated circuit, such as an Application Specific Integrated Circuit (ASIC), Field Programmable Gate Array (FPGA) etc. The memory 604 may include one or several types of memory suitable for the processor, such as read-only memory (ROM), random-access memory, cache memory, flash memory devices, optical storage devices, solid state disk, hard disk drive, etc.

In some examples as discussed above, the processing node may be incorporated into a treatment apparatus, and examples of the present disclosure also provide a treatment apparatus comprising either or both of a processing node and/or a training node as discussed above, and/or a planning node operable to implement a method for developing and/or adapting a radiotherapy treatment plan.

FIGS. 1 to 4 discussed above provide an overview of methods which may be performed according to different examples of the present disclosure. These methods may be performed by a training node and a processing node respectively, as illustrated in FIGS. 5 and 6. As discussed above, an implementation of the methods presented herein, Noise2Aliasing, has been validated on clinical data, and experimental results for this implementation are presented below, demonstrating the effects and advantages discussed above.

### Experiments

The SPARE Varian® dataset was initially used to study whether Noise2Aliasing could match the performance of the supervised baseline and whether it could outperform the baseline when noise was added to the projections. An internal Elekta® data set was then used to explore the requirements for the method to be applied to an existing clinical dataset. These experiments required around 64 GPU days on NVIDIA A100 GPUS.

Training of the model was performed on 2D slices. The projections obtained during a scan can be sub-sampled according to the pseudo-average subset selection method and then used to obtain respiratory uncorrelated 3D reconstructions. In Noise2Aliasing these are used for both input and target during training. Given two volumes (x, y), the training pairs (xᵢ_{^{(k)}},yᵢ_{^{(k)}}) are the same i-th slice along the k-th dimension of each volume chosen to be the axial plane.

The two datasets used in the study were:
1. The SPARE Varian® dataset was used to provide performance results on publicly available patient data. To more closely resemble normal respiratory motion per projection image, the 8 minutes scan was used from each patient (five such scans are available in the dataset). Training was performed over 4 patients with 1 patient used as a test set. The hyperparameters were optimized over the training dataset.
2. An internal Elekta® dataset (IRB approved) of 30 lung cancer patient 4DCBCTs from 2020 to 2022 was used. The 4DCBCTs were originally used for IGRT. Training was performed over 25 patients, with 5 patients for testing. The scans are 4 minute, 205° scans with a 120 keV source and 512×512 sized detector, using Elekta® LINACs. The data were anonymized prior to analysis.

Projection noise was added using the Poisson distribution to the SPARE Varian® dataset, in order to evaluate the ability of the unsupervised method to reduce it. Given a projected value of p and a photon count π (chosen to be 2500), the rate of the Poisson distribution is defined as πe^{-p} and given a sample q from this distribution, then the new projected value is

$$\tilde{p} = - log ( \frac{q}{π} ) .$$

The architecture used for experimental validation of Noise2Aliasing was the Mixed Scale Dense CNN (MSD), the most successful architecture from Noise2Inverse. The MSD makes use of dilated convolutions to process features at all scales of the image. The experimentation used the MSD with depth 200 and width 1, Adam optimizer, MSE loss, a batch size of 16, and a learning rate of 0.0001.

Two baselines were used for comparison. The first baseline was the traditional FDK obtained using RTK. The second baseline was the supervised approach, in which the model architecture was replaced with the MSD to enable a fair comparison. In the supervised approach, the model was trained by using reconstructions obtained from subsets defined with pseudo-average subset selection as input, and using as target output reconstructions obtained from all available projections.

The metrics used for Analysis of the performance of Noise2Aliasing against the baselines were the Root Mean Squared Error (RMSE), Peak Signal-to-Noise Ratio (PSNR), and Structural Similarity Index Measure (SSIM). All the metrics are defined between the output of the neural network and a 3D (CB) CT scan. For the SPARE Varian® dataset, the Regions of Interest (ROIs) were used as defined and provided, and the 3D reconstruction using all the projections available was used as a ground truth. For the internal Elekta® dataset, the planning CT was deformed to each of the phases reconstructed using the FDK algorithm, and the metric was evaluated over only the 4DCBCT volume boundaries.

### Results and Discussion

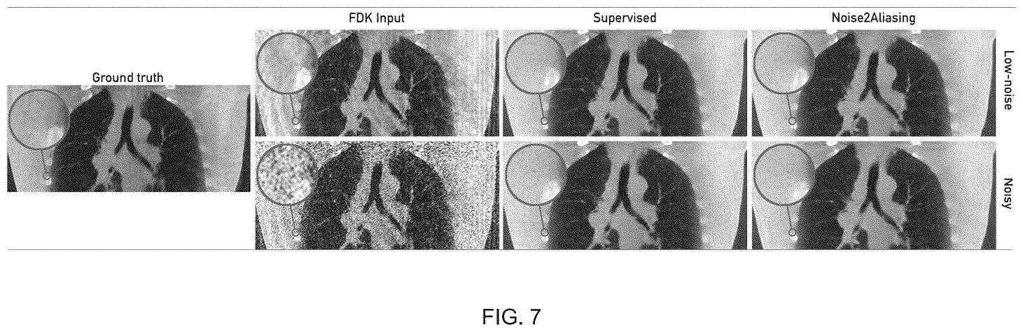

FIG. 7 illustrates a qualitative comparison between methods using a coronal view of the patient in the test set. Noise2Aliasing and the Supervised method produce very similar images in the low-noise case. With noisy data, the supervised method tends to re-create the noise seen during training.

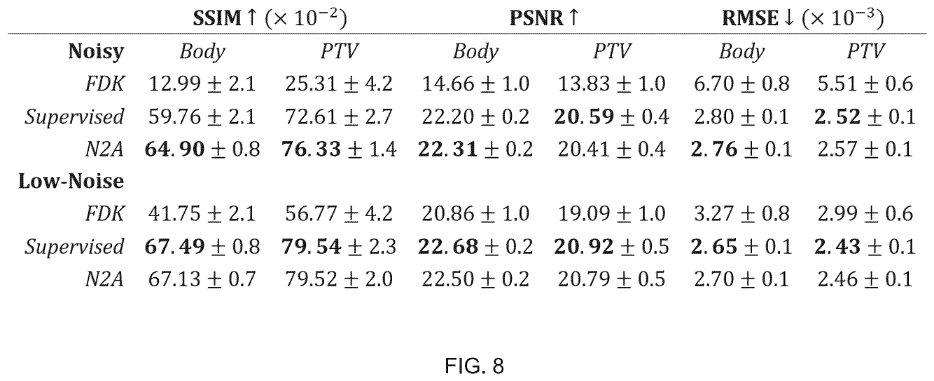

FIG. 8 illustrates Table 1, which shows metrics for the comparison between FDK, the Supervised method, and Noise2Aliasing (N2A). Values are mean and standard deviation computed across all phases of patient 1 of the SPARE Varian dataset. The Planned Target Volume (PTV) ROI is less affected by noise compared to the whole Body, which is what causes the supervised model to outperform N2A in terms of PSNR and RMSE.

### Performance on SPARE Varian

Inference speed when using a batch size of 16 with the NVIDIA A100 GPU averages 600 ms per volume made of 220 slices. A qualitative evaluation of the method is illustrated in FIG. 7, in which Noise2Aliasing can be seen to match the visual quality of the supervised approach on the low-noise dataset on both soft tissue and bones. The metrics summarized in Table 1 (FIG. 8) show the mean and standard deviation across all phases for a single patient. In the low-noise setting, both supervised and Noise2Aliasing outperform FDK, with very similar results, often within a single standard deviation.

Noise2Aliasing consequently successfully matches the performance of the supervised baseline.

### Performance on Noisy SPARE Varian

From FIG. 7 and Table 1, the supervised approach can be seen to reproduce the noise that was seen during training, while Noise2Aliasing manages to remove it consistently. Noise2Aliasing outperforms the supervised approach, especially in the soft tissue area around the lungs, where the noise has the greatest effect on the attenuation coefficients.

Noise2Aliasing can be seen to reduce the artifacts present in reconstructions caused by stochastic noise in the projections used, outperforming the supervised baseline.

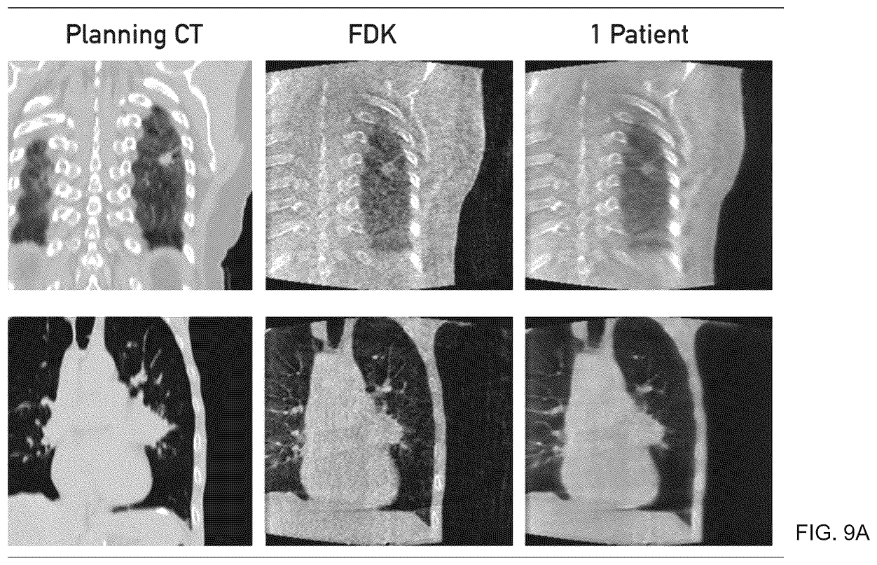

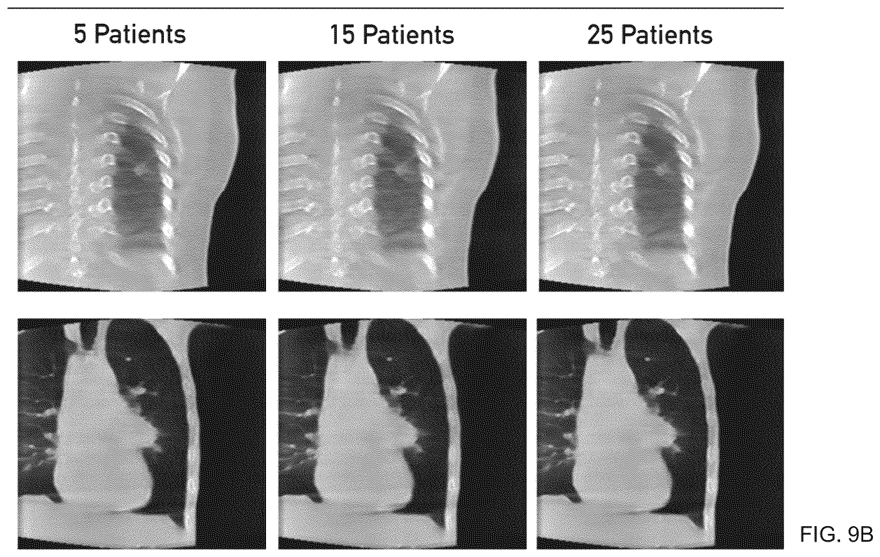

FIGS. 9A and 9B illustrate illustrates comparison of performance of Noise2Aliasing with different-sized datasets from the internal Elekta® dataset. With fewer patients, the model is more conservative and tends to keep more noise, but also smudges the interface between tissues and bones. With more patients, more of the view-aliasing is addressed, and the reconstruction is sharper, however, a few small anatomical structures tend to be suppressed by the model.

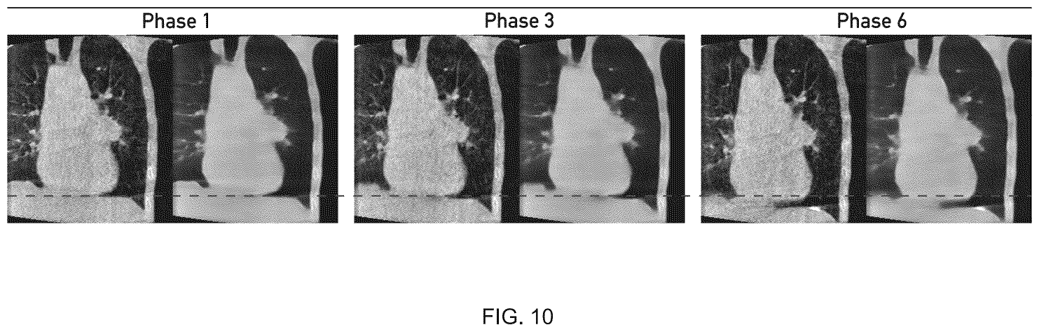

FIG. 10 indicates that motion extent is accurately resolved by Noise2Aliasing when using 25 patients from the internal Elekta® dataset. On the left of each phase in the image is the FDK reconstruction, while on the right is the output of the model.

### Performance on the Internal Dataset

Noise2Aliasing achieves a median relative improvement of 2.4% PSNR, 4.6% RMSE, and 32% SSIM relative to the FDK reconstruction when using the entire training set. Additionally, from FIG. 10 it can be seen how the breathing extent is matched with sharp reconstruction of the diaphragm. Overall, using more patients results in better noise reduction and sharper reconstructions (see FIGS. 9A and 9B), especially between fat tissue and skin and around the bones. However, the model also tends to remove small anatomical structures as high-frequency objects that cannot be distinguished from the noise.

When applied to a clinical dataset, Noise2Aliasing benefits from more patients being included in the dataset, however, qualitatively good performance is already achieved with 5 patients. No additional data collection was required, and the method can be applied without major changes to the current clinical practice.

The above discussion demonstrates that example methods according to the present disclosure are provably able to reduce both view-aliasing and stochastic projection noise from 4DCBCTs using an unsupervised deep learning method. Respiratory uncorrelated input and target output reconstructed volumes are used to train an unsupervised ML model to reduce both noise and aliasing artefacts in 4D medical images. An implementation of the methods disclosed herein has been shown to outperform a supervised approach when stochastic noise is present in the projections, and to match performance on a popular benchmark. The methods disclosed herein can be trained on existing historical datasets, and do not require changes to current clinical practices.

The methods of the present disclosure may be implemented in hardware, or as software modules running on one or more processors. The methods may also be carried out according to the instructions of a computer program, and the present disclosure also provides a computer readable medium having stored thereon a program for carrying out any of the methods described herein. A computer program embodying the disclosure may be stored on a computer readable medium, or it could, for example, be in the form of a signal such as a downloadable data signal provided from an Internet website, or it could be in any other form.

It should be noted that the above-mentioned examples illustrate rather than limit the disclosure, and that those skilled in the art will be able to design many alternative embodiments without departing from the scope of the appended claims or numbered embodiments. The word “comprising” does not exclude the presence of elements or steps other than those listed in a claim or embodiment, “a” or “an” does not exclude a plurality, and a single processor or other unit may fulfil the functions of several units recited in the claims or numbered embodiments. Any reference signs in the claims or numbered embodiments shall not be construed so as to limit their scope.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third.” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

What is claimed is:

1. A computer implemented method for training an unsupervised Machine Learning (ML) model to reduce noise and aliasing artefacts in a reconstructed four-dimensional (4D) medical image of a patient, wherein the ML model comprises a plurality of trainable parameters, and wherein the method comprises:
i. obtaining a projection set comprising a plurality of two-dimensional (2D) projections of a training patient volume, wherein the obtained 2D projections represent a plurality of phases of a respiratory cycle of the training patient, and wherein the obtained 2D projections comprise measurement noise;
ii. repeating, for a plurality of iterations:
selecting two non-empty, mutually disjoint subsets that form a partition of the projection set, wherein each of the two selected subsets contains 2D projections that are respiratory uncorrelated;
for each of the two selected subsets, using a linear reconstruction algorithm to reconstruct a volumetric image of the training patient from the 2D projections contained in the subset; and
adding the reconstructed volumetric images of the patient to a training data set as an input volume and corresponding target output volume training pair; and
wherein the method further comprises:
iii. using training pairs from the training data set to update values of the trainable parameters of the ML model.

2. The computer implemented method as claimed in claim 1, further comprising:
repeating i. and ii. for a plurality of training patients.

3. The computer implemented method as claimed in claim 1, wherein the obtained 2D projections comprise stochastic measurement noise.

4. The computer implemented method as claimed in claim 1, wherein the obtained 2D projections comprise measurement noise that is elementwise independent and mean-zero.

5. The computer implemented method as claimed in claim 1, wherein the ML model is a Convolutional Neural Network (CNN).

6. The computer implemented method as claimed in claim 1, wherein for each of the two selected subsets, using a linear reconstruction algorithm to reconstruct a volumetric image of the training patient from the 2D projections contained in the subset comprises using an analytic reconstruction algorithm to reconstruct the volumetric image.

7. The computer implemented method as claimed in claim 1, wherein for each of the two selected subsets, using a linear reconstruction algorithm to reconstruct a volumetric image of the training patient from the 2D projections contained in the subset comprises:
using an algorithm having a property that an average of reconstructions generated by the algorithm using subsets of an available projection set is approximately equal to the reconstruction generated by the algorithm using all projections of the available projection set.

8. The computer implemented method as claimed in claim 1, wherein selecting two non-empty, mutually disjoint subsets that form a partition of the projection set, wherein each of the two selected subsets contains 2D projections that are respiratory uncorrelated, comprises:
using a sampling pattern to select projections for inclusion in each of the two subsets, wherein the sampling pattern is the same as a sampling pattern used to select projections for inclusion in respiratory correlated sets for reconstruction of a 4D medical image of a patient.

9. The computer implemented method as claimed in claim 1, wherein using training pairs from the training data set to update values of the trainable parameters of the ML model comprises repeating, until a convergence condition is satisfied:
inputting an input of a training pair from the training data set to the ML model, wherein the ML model processes the input in accordance with current values of the trainable parameters of the ML model and generates an ML model output;
comparing the ML model output to the target output of the training pair; and
updating trainable parameters of the ML model to optimize a function of the comparison.

10. The computer implemented method as claimed in claim 1, wherein using training pairs from the training data set to update values of the trainable parameters of the ML model comprises:
using corresponding slices from corresponding dimensions of each of the input and target output volumes.

11. A computer implemented method for reducing noise and aliasing artefacts in a reconstructed four-dimensional, 4D, medical image of a patient, the method comprising:
obtaining a projection set comprising a plurality of two-dimensional (2D) projections of a patient volume, wherein the obtained 2D projections represent a plurality of phases of a respiratory cycle of the patient, and wherein the obtained 2D projections comprise measurement noise;
dividing the plurality of 2D projections in the projection set into a plurality of phase sets, wherein each phase set comprises 2D projections that are respiratory correlated, corresponding to a single phase of the respiratory cycle of the patient;
for each phase of the respiratory cycle of the patient, generating a reconstructed volumetric image of the patient from the 2D projections contained in the corresponding phase set; and
inputting the generated reconstruction to an unsupervised Machine Learning (ML) model, wherein the ML model is operable to process the input generated reconstructed volumetric images according to trained parameters of the ML model and to output volumetric images of the patient having reduced noise and aliasing artefacts, and wherein the ML model is trained using reconstructed volumetric images that are respiratory uncorrelated.

12. The computer implemented method as claimed in claim 11, wherein the ML model is trained using training pairs of input and target output reconstructed volumetric images, and wherein both the input and target output reconstructed volumetric images of a training pair are respiratory uncorrelated.

13. The computer implemented method as claimed in claim 12, wherein the input and target output reconstructed volumetric images of a training pair are generated from two non-empty, mutually disjoint subsets that form a partition of a projection set for a training patient, and wherein each of the two mutually disjoint subsets contains 2D projections that are respiratory uncorrelated.

14. The computer implemented method as claimed in claim 11, wherein the obtained 2D projections comprise stochastic measurement noise.

15. The computer implemented method as claimed in claim 11, wherein the obtained 2D projections comprise measurement noise that is elementwise independent and mean-zero.

16. The computer implemented method as claimed in claim 11, wherein the ML model is a Convolutional Neural Network (CNN).

17. The computer implemented method as claimed in claim 11, wherein dividing the plurality of 2D projections in the projection set into a plurality of phase sets, wherein each phase set comprises 2D projections that are respiratory correlated, corresponding to a single phase of the respiratory cycle of the patient, comprises:
using a sampling pattern to select projections for inclusion in each of the phase sets; and wherein the sampling pattern is the same as a sampling pattern used to select projections for inclusion in respiratory uncorrelated sets for training of the ML model.

18. The computer implemented method as claimed in claim 11, wherein inputting the generated reconstruction to an ML model comprises:
inputting slices from the generated reconstruction to the ML model.

19. The computer implemented method as claimed in claim 11, wherein the ML model is trained by:
i. obtaining a projection set comprising a plurality of two-dimensional (2D) projections of a training patient volume, wherein the obtained 2D projections represent a plurality of phases of a respiratory cycle of the training patient, and wherein the obtained 2D projections comprise measurement noise;
ii. repeating, for a plurality of iterations:
selecting two non-empty, mutually disjoint subsets that form a partition of the projection set, wherein each of the two selected subsets contains 2D projections that are respiratory uncorrelated;
for each of the two selected subsets, using a linear reconstruction algorithm to reconstruct a volumetric image of the training patient from the 2D projections contained in the subset; and
adding the reconstructed volumetric images of the patient to a training data set as an input volume and corresponding target output volume training pair; and
iii. using training pairs from the training data set to update values of one or more trainable parameters of the ML model.

20. A radiotherapy treatment apparatus comprising at least one of:
a) a training node for training an unsupervised Machine Learning (ML) model to reduce noise and aliasing artefacts in a reconstructed four-dimensional (4D) medical image of a patient, wherein the ML model comprises a plurality of trainable parameters, the training node comprising processing circuitry configured to cause the training node to:
i. obtain a projection set comprising a plurality of two-dimensional, 2D, projections of a training patient volume, wherein the obtained 2D projections represent a plurality of phases of a respiratory cycle of the training patient, and wherein the obtained 2D projections comprise measurement noise;
ii. repeat, for a plurality of iterations:
selecting two non-empty, mutually disjoint subsets that form a partition of the projection set, wherein each of the two selected subsets contains 2D projections that are respiratory uncorrelated;
for each of the two selected subsets, using a linear reconstruction algorithm to reconstruct a volumetric image of the training patient from the 2D projections contained in the subset; and
adding the reconstructed volumetric images of the patient to a training data set as an input volume and corresponding target output volume training pair; and
wherein the processing circuitry is further configured to cause the training node to:
iii. use training pairs from the training data set to update values of the trainable parameters of the ML model; or
b) a processing node for reducing noise and aliasing artefacts in a reconstructed four-dimensional, 4D, medical image of a patient, the processing node comprising processing circuitry configured to cause the processing node to:
obtain a projection set comprising a plurality of two-dimensional (2D) projections of the patient volume, wherein the obtained 2D projections represent a plurality of phases of a respiratory cycle of the patient, and wherein the obtained 2D projections comprise measurement noise;
divide the plurality of 2D projections in the projection set into a plurality of phase sets, wherein each phase set comprises 2D projections that are respiratory correlated, corresponding to a single phase of the respiratory cycle of the patient;
for each phase of the respiratory cycle of the patient, generate a reconstructed volumetric image of the patient from the 2D projections contained in the corresponding phase set; and
input the generated reconstruction to an unsupervised Machine Learning (ML) model, wherein the ML model is operable to process the input generated reconstructed volumetric images according to trained parameters of the ML model, and to output volumetric images of the patient having reduced noise and aliasing artefacts, wherein the ML model is trained using reconstructed volumetric images that are respiratory uncorrelated.
